(19) United States
(12) Patent Application Publication
(10) Pub. No.: US 2012/0156670 A1
(43) Pub. Date: Jun. 21, 2012

(54) IN VITRO UROGENITAL CO-CULTURE MODELS

(75) Inventors: Ayesha MAHMOOD, Orlando, FL (US); Melissa M. HERBST-KRALOVETZ, Phoenix, AZ (US); William WARREN, Orlando, FL (US)

(73) Assignees: ARIZONA STATE UNIVERSITY, Tempe, AZ (US); SANOFI PASTEUR VAXDESIGN CORP., Orlando, FL (US)

(21) Appl. No.: 13/296,695

(22) Filed: Nov. 15, 2011

Related U.S. Application Data
(60) Provisional application No. 61/413,698, filed on Nov. 15, 2010.

(51) Int. Cl. C12Q 1/70; C12Q 1/02

(52) U.S. Cl. 435/5; 435/29

(57) ABSTRACT

The invention is directed to co-culture systems comprising (i) rotating wall vessel (RWV)-cultured epithelial or differentiated tissue attached to microcarrier beads and (ii) the peripheral tissue equivalent (PTE) module of the MIMIC® system, and to methods of using the co-culture systems for assessing chemical or biological (bacterial or viral) insults. The system models mucosal exposure to chemicals, pathogens or antigen at various sites in the human body. The microcarrier and MIMIC® co-culture approach provides an in vitro co-culture model that simultaneously demonstrates mucosa-mediated antigen presentation and immunogenic responses. Models of the present invention can be used, for example, in assessments of disease pathogenesis and in pharmaceutical development, reproductive physiology, and immunological and toxicological evaluations. Models of the present invention can generate patient-specific localized mucosal immunology using primary cells, resembling the human physiological situation.

### CROSS-REFERENCE TO RELATED APPLICATIONS

This application claims benefit under 35 U.S.C. §119(e) of U.S. Patent Application No. 61/413,698, filed Nov. 15, 2010, which is hereby incorporated herein by reference in its entirety.

### BACKGROUND TO THE INVENTION

Three-dimensional (3D) tissue models offer great potential in generating in vitro human functional tissue with similarities to the native in vivo environment. Methods for such systems generally include the use of extracellular matrix (ECM) materials, modified culture media nutrients, and static and/or flow conditions to mimic the 3D physiological environment for nutrient transport.

In addition to direct explants or single-cell cultures, some of these methods include the use of cellular aggregates, which can also serve as microcarriers to maximize the cell loading capacity, with limited anatomical replication of physiological tissue. As an example, rotating-wall vessel (RWV) bioreactors have been used successfully for cell and tissue culture in a microgravity environment. These culture systems promote nutrient distribution under low-shear fluid dynamics (Hammond, 2001). The RWV bioreactor has been used to generate a variety of 3D tissues. Examples include small intestine (Goodwin, 1993), prostate (O'Conner, 1999), ovarian (Goodwin, 1997), vaginal (Hjelm, 2010), hepatocytes (Mitteregger, 1999), and chondrocytes (Duke, 1993).

In vitro microcarrier cultures of vaginal epithelial tissue aggregates cultured in the RWV bioreactor possess a stratified squamous epithelium that is differentiated, contains microridges and produces mucus in a physiologically relevant manner (Hjelm, 2010). These RWV-derived vaginal epithelial tissue aggregates have been shown to express estrogen and progesterone receptors and these receptors are functional in response to hormone stimulation (unpublished findings). These systems have shown improved host surface recognition and propagation for both bacterial and viral infections and drug development (Mikaty, 2009; Andrei, 2006).

However, explant culture of tonsilar lymphoid tissue (Nauman, 2007; Hammond, 2001) or lymphoid cells under microgravity conditions show loss of T and B cell viability and function over long-term culture. These culture systems show lack of immune cell activation, along with reduced cytokine production and antibody responses. Similar findings of compromised lymphocyte function under microgravity conditions have been reported in space flight travel, from ex vivo cultures of blood drawn from astronauts, and in RWV bioreactor in vitro systems (Simons, 2006; Borchers, 2002; Cogoli, 2009; Ritz, 2006; Fitzgerald, 2009).

### In Vitro Urogenital Models

Urinary tract infections are common infectious diseases. Susceptibility to acquiring UTIs has been linked to gender, age, genetic factors (non-secretor status, ABO blood-group antigen), biological factors (congenital abnormalities, urinary obstruction, diabetes, STDs, incontinence, recurrent infection), along with hygienic practices and sexual activity (Foxman, 1995; 2002).

In total, 80-90% of urinary tract infections are caused by E. coli (Funfstuck, 2006) and over the past two decades there has been an increase in reported cases of antimicrobial treatment-resistant E. coli strains in urinary tract infections (Aboderin, 2009). UTI infections have been linked to infertility through reproductive tract infections (Pallati, 2008; Foxman, 2002), pylonephritis or renal disease (Funfstuck, 2006), and prostatitis (Lipsky, 1999; Weidner, 2003) in the male population.

Additionally, sexually transmitted infections (STI) have reached epidemic proportions. Many of these STI can act as predisposing factors for contracting HIV and other STI (Rebbapragada et al, 2007). Healthy intact vaginal epithelium provides a barrier to STI; however, external insults (e.g., pathogens or chemical) can disrupt this barrier and result in increased susceptibility to infection.

The vaginal mucosa is considered a primary site of the immune response and antigen recognition/APC priming, eventually leading to B and T cell stimulation in the draining lymph nodes. The specialized functionality of various regions of the female reproductive mucosa can be attributed to the degree of close contact with the external environment and its constant exposure to environmental irritants and pathogens (Kiyono, 1995).

The development of vaginally-applied microbicides to reduce or prevent sexually transmitted diseases has led to detergent (e.g., nonoxynol-9 (N-9), BufferGel), polyanion (e.g., napthelene sulfonated PRO 2000), polysaccharide- and protein-based natural and synthetic formulations (e.g., carregeenan, a sulfonated galactan, TLR agonists) with limited success in clinical trials. To date, no U.S. FDA-approved microbicide is on the market.

The toxic effects of the most common surfactant-based microbicide, nonoxynol-9, have been widely reported over the past decade. Direct or non-specific binding of this microbicide to the epithelia is designed to prevent pathogen attachment and infection prevention. However, the repeated use of microbicides has not been studied long-term and most clinical trials have been limited and inconclusive (Lederman, 2006).

In vitro culture systems of vaginal epithelial cells treated with N-9 show increased inflammation, cell death, and damaged tissue mucosa. These findings suggest that the use of this particular surfactant-based microbicide may, in fact, increase the risk of infection. Numerous in ex vivo, in vitro, and in vivo findings suggest a correlation between the use of N-9 microbicide and damaged reproductive tract epithelia (Cone, 2006; Dayal, 2006).

To date, there has been no report of an in vitro culture system that mimics the effects of toxic microbicides on human female reproductive mucosa and immune cells, to generate localized immune responses. Thus, there is a continuing need for in vitro model systems with those characteristics, based on human female reproductive mucosa and immune cells, without the addition of exogenous factors to promote growth or cell function, that can be used, for example, for screening candidate microbicides.

### SUMMARY OF THE INVENTION

The present invention is directed to methods for assaying responses of cells found in the female reproductive mucosa to different test agents. The methods utilize a novel in vitro co-culture model, under static or non-static conditions, that comprises both cells of the reproductive mucosa and immune cells. One component of the co-culture of the present invention uses RWV-cultured epithelial or differentiated tissue attached to microcarrier beads that is subjected to chemical or biological (bacterial or viral), inter alia, insult under microgravity or non-microgravity conditions. The primed cell-bearing microcarrier beads are then introduced to the second component, a peripheral tissue equivalent (PTE) culture, to form a co-culture, under static or non-static conditions, in which immunological responses are generated that, in sum, replicate mucosal exposure to chemicals, pathogens, or antigens at various sites in the human body. The co-culture environment further addresses the adverse effects of shear on lymphocytes, a known shortcoming of the RWV culture system, through the use of the microcarrier beads (Chemy, 1987).

As described in U.S. Pat. Nos. 7,785,883, 7,771,999, 7,709,256, 7,709,257, and 7,855,074, Sanofi Pasteur VaxDesign's MIMIC® system comprises a peripheral tissue equivalent (PTE) culture module and a lymphoid tissue equivalent (LTE) culture module and, optionally, a disease tissue culture module (DM). The PTE module generates antigen-presenting cells by natural extravasation through a confluent layer of human umbilical vascular endothelial cells (HUVECs) without the addition of exogenous factors. In addition to the generation of dendritic cells, the system can also have macrophages in the supporting extracellular matrix layer below the endothelium. These specialized cells have the ability to pick up antigen and generate a localized immune response. As described herein, Sanofi Pasteur VaxDesign's MIMIC® system was used to generate dendritic cells and used to evaluate the antigen-presenting function of RWV-cultured microcarriers of female reproductive tract epithelium.

Transitioning from innate to adaptive immune responses requires the use of the LTE module, where T and B cells are co-cultured with the DCs from the PTE module. Immune responses from the MIMIC® system have been shown to correlate with a number of findings relating to innate and adaptive immune response to adjuvants, vaccines, nanoparticles, and various pathogens (Higbee, 2009; Ma, 2010; Byers, 2009). The LTE module may be used to further characterize the PTE-derived dendritic cells produced using the methods of the present invention.

The microcarrier and MIMIC® co-culture approach addresses the lack of an in vitro culture model that simultaneously demonstrates mucosa-mediated antigen presentation and immunogenic responses. Models of the present invention can be used, for example, to assess disease pathogenesis and pharmaceutical development, reproductive physiology, along with tissue, immunological, and toxicological evaluations. In essence, this approach can generate patient-specific localized mucosal immunology using primary cells, resembling the human physiological situation.

In particular, the methods of the present invention are generally directed to assaying a cellular response to a test agent in an in vitro urogenital co-culture through the steps of: a) priming cell-bearing microcarrier beads with one or more test agents; b) adding the primed cell-bearing microcarrier beads to a peripheral tissue equivalent (PTE) culture, thereby preparing a co-culture; and c) assaying a cellular response to the test agent in the co-culture, thereby assaying a cellular response to a test agent in an in vitro urogenital co-culture.

In a particular embodiment, the present invention is directed to a method of assaying a cellular response to a test agent in an in vitro urogenital co-culture, said method comprising: a) priming epithelial cell-bearing microcarrier beads with one or more test agents; b) adding the primed microcarrier beads of a) to a peripheral tissue equivalent (PTE) culture, thereby preparing a co-culture, wherein the PTE culture comprises primary human umbilical vascular endothelial cells (HUVECs) attached to a substantially planar matrix and a population of monocytes of varying maturation states; and c) assaying a cellular response to the test agent in the co-culture of b), thereby assaying a cellular response to a test agent in an in vitro urogenital co-culture.

In certain aspects of this embodiment, the epithelial cell-bearing microcarrier beads are (i) vaginal epithelial cell-bearing microcarrier beads, or (ii) endocervical epithelial cell-bearing microcarrier beads. In particular aspects, the microcarrier beads are dextran beads, and include collagen-coated dextran beads.

In certain aspects of this embodiment, the co-culture of b) is prepared under static conditions. In other aspects, the co-culture of b) is prepared under non-static conditions, such as, but not limited to, rocking of the co-culture.

In certain aspects of this embodiment, the one or more test agents are selected from the group consisting of bacteria, viruses, environmental pollutants, vaccines, adjuvants, immunotherapy candidates, cosmetics, drugs, biologics, proteins, and chemical compounds. In a particular aspect, the test agent is a species or strain of bacteria, the test agent is a chemical compound containing nonoxynol-9, or the test agent is a species or strain of bacteria and a chemical compound containing nonoxynol-9.

In certain aspects of this embodiment, the matrix is selected from the group consisting of collagen, a collagen gel, a hydrogel, PLA, PLGA, gelatin, hyaluronic acid, a synthetic material, and a combination thereof. In a particular, aspect, the matrix is a collagen gel, and optionally further comprising one or more cell adhesion proteins selected from the group consisting of fibronectin, vitronectin, and laminin. In further aspects, the collagen gel may comprise about 4% collagen.

In certain aspects of this embodiment, the cellular response is a parameter selected from the group consisting of cellular maturation, cellular growth rate, cell number, apoptosis, cytokine production, chemokine production, and cellular marker expression. In one aspect, the cellular response is cellular maturation or cytokine production, or both. In a particular aspect, the cellular response is cellular maturation and the cellular maturation is maturation of dendritic cells present in the population of monocytes of varying maturation states. The maturation of the dendritic cells may be assayed by screening for expression of one or more dendritic cell maturation markers selected from the group consisting of HLA-DR, CD14, CD86, and CCR7. In a further particular aspect, the cellular response is cytokine production and cytokine production is production of one or more cytokines selected from the group consisting of IL-1a, IL-1b, IL-6, IL-8, IL-10, and TNF-α.

In certain aspects of this embodiment, epithelial cell-bearing microcarrier beads are prepared in a rotating-wall vessel bioreactor.

In one aspect of this embodiment, the cells of the PTE culture consist of HUVECs and a population of monocytes of varying maturation states.

In certain of the aspects of this embodiment, the population of monocytes of varying maturation states is a population of monocytes that expresses a detectable level of one or more of the dendritic cell maturation markers selected from the group consisting of HLA-DR, CD14, CD86, and CCR7.

In this embodiment and all of its aspects, the method may further comprise comparing the response assayed in c) to the response assayed in a counterpart control co-culture in which the microcarrier beads were not primed with the one or more test agents.

### BRIEF DESCRIPTION OF THE FIGURES

FIG. 1. Co-culture module of primed microcarriers in the MIMIC® system. The effects of bacterial antigen and a microbicide compound were evaluated by phenotypic analysis of dendritic cell maturation markers and cytokine production in culture supernatants. Left. Schematic view of culture conditions. Right. Phase contrast image of differentiated vaginal epithelial microcarriers on top of the PTE module.

FIG. 2. Total immune cell recovery calculated by trypan blue exclusion and total DCs per treatment were calculated by applying the percentage marker expression from flow cytometry data. The trends showed that total DC viability was reduced with the introduction of N-9 and E. coli bioparticle treatment to the PTE antigen-presenting cells. The vaginal epithelium in a model of the present invention showed improved antigen-presenting characteristics, based on the percentage conversion to HLA-DR-bright and loss of CD14 marker expression, classic DC maturation profiles. CD86 and CCR7 expression in the vaginal model also confirm the antigen uptake/presentation responsiveness of the vaginal model.

FIG. 3. Pro-inflammatory cytokine production in the female reproductive mucosal models. Overall cytokine levels in the vaginal model were several times higher than in the endocervical model. However, the endocervical model was more sensitive to E. coli and N-9.

FIG. 4. Overall, cytokine levels for the vaginal model were several fold-higher than in the endocervical model. Similar to the pro-inflammatory cytokines profiles, the endocervical model showed a more consistent response to E. coli and N-9 above the no treatment microcarrier plus PTE control. Interleukin-10 and TNF-α up-regulation after bacterial infection was consistent with other reported findings (Basset, 2003). No significant change in the interferon-γ levels is consistent with the purity of the APCs in the reverse-transmigrated fraction, with less than 1% contaminating T cells. Both systems showed up-regulation of GM-CSF and MIP-1α.

### DETAILED DESCRIPTION OF THE INVENTION

As stated above, and described in detail below, the present invention is directed to methods for assaying responses of cells found in the female reproductive mucosa to different test agents. The methods utilize a novel in vitro co-culture model that comprises both cells of the reproductive mucosa and immune cells. The methods do not require the addition of exogenous factors to promote growth or cell function. The methods can be used, inter alia, to assay cellular responses to particular insults (e.g., pathogens or chemical) that might be encountered by the reproductive mucosa.

The methods of the present invention are generally directed to assaying a cellular response to a test agent in an in vitro urogenital co-culture through the steps of: a) priming cell-bearing microcarrier beads with one or more test agents; b) adding the primed cell-bearing microcarrier beads to a peripheral tissue equivalent (PTE) culture, thereby preparing a co-culture; and c) assaying a cellular response to the test agent in the co-culture, thereby assaying a cellular response to a test agent in an in vitro urogenital co-culture.

The cells used to prepare the cell-bearing microcarrier beads are generally any cell type of the female reproductive mucosa that is amenable to culture and growth on microcarrier beads. The cells may be, for example, primary cells, cells isolated from tumors (both immortal and non-immortal), and cells from cultured cell lines. Exemplary cell types include vaginal epithelial cells and endocervical epithelial cells. However, additional cell types that may be used in other embodiments include ectocervical epithelial cells, ovarian epithelial cells, Fallopian tube epithelial cells, bladder and uterine epithelial cells. Particular cell-bearing microcarrier beads used in methods of the present invention include, but are not limited to, vaginal epithelial cell-bearing microcarrier beads and endocervical epithelial cell-bearing microcarrier beads.

The microcarrier beads that are used to prepare the cell-bearing microcarrier beads are any beads that permit the attachment and growth of the cell types used in the methods of the present invention. Dextran micro-carrier beads, such as Cytodex®, beads from Sigma, Solohill, or GE are particularly suitable, but other beads include plastic beads, with or without collagen coating. The size of the beads may vary depending on the particular circumstances of the method being performed, but suitable bead sizes will generally range from about 10 to 500 microns, and includes beads ranging in size between 50 and 100 microns. The microcarrier beads may be coated with collagen prior to culturing with cells as a means of increasing the surface area of the beads and providing extracellular matrix necessary for the differentiation of in vitro-grown epithelium.

The cell-bearing microcarrier beads may be produced using any of the conventional techniques known in the art of tissue culture. An exemplary technique that has resulted in the production of suitable cell-bearing microcarrier beads is preparation of the cell-bearing microcarrier beads in rotating-wall vessel bioreactor.

In certain embodiments, the cell-bearing microcarrier beads will have been treated with or exposed to one or more test antigens. Such cell-bearing microcarrier beads are generally described as “primed” cell-bearing microcarrier beads herein. The test agents that are used in the methods of the present invention are generally unlimited in scope or character. The test agents need only be a substance, compound, molecule, etc., amenable for evaluation in the methods of the invention. Test agents include bacteria, viruses, environmental pollutants, vaccines, adjuvants, immunotherapy candidates, cosmetics, drugs, biologics, proteins, and chemical compounds. In certain embodiments of the invention, the test agent is a species or strain of bacteria. In other embodiments of the invention, the test agent is virus, such as human papillomavirus. In still other embodiments, the test agent is a chemical compound, such as a compound containing nonoxynol-9. In further embodiments, two or more test agents are evaluated. For example, both a species or strain of bacteria and a chemical compound can be evaluated, or both virus and a chemical compound can be evaluated. For example, the epithelial cell-bearing microcarrier beads may be exposed to the chemical compound, such as a compound containing nonoxynol-9, for a period of time, and then cultured in the presence of a species or strain of bacteria or a virus.

The human immune system has two arms: (1) an innate response, which reacts within minutes through days, and (2) an adaptive response, which reacts within one to two weeks. The peripheral tissue equivalent (PTE) culture used in the methods of the present invention simulates the innate immune response that occurs in the peripheral tissues of the body, such as the skin, lungs, and other mucosal tissues that may become exposed to pathogens in the environment. PTE cultures can be used to predict adjuvant and vaccine potency, toxicity, and other desired (and undesired) effects prior to the initiation of animal studies or clinical trials. Sanofi Pasteur VaxDesign has developed different versions of PTE cultures, termed “PTE modules”, in the MIMIC® system that includes a Transwell®-based PTE culture that efficiently generates antigen-presenting cells such as dendritic cells.

The PTE cultures used in the methods of the present invention are constructed by layering endothelial cells over a matrix that has been prepared in a Transwell® device. Suitable matrices include those comprising collagen, a collagen gel, a hydrogel, PLA, PLGA, gelatin, hyaluronic acid, a synthetic material, and a combination thereof. In one embodiment, the matrix is a collagen matrix. In another embodiment, the matrix is a collagen gel matrix, such as, but not limited to, a collagen gel comprising 4% collagen. Each of the matrices used in the PTE cultures of the present invention may optionally further comprise one or more cell adhesion proteins, including, but not limited to, fibronectin, vitronectin, and laminin. Suitable endothelial cells include primary human umbilical vascular endothelial cells (HUVECs). Upon establishment of a confluent layer of endothelial cells on the collagen matrix, a population of immune cells in nutrient media is added to the construct. Suitable populations of immune cells include monocytes, such as CD33⁺ monocytes, as well as peripheral blood mononuclear cells (PBMCs).

Over a period of hours, monocytes present in the population of immune cells travel through the endothelial cell layer and into the collagen matrix. The nutrient media containing the remaining monocytes is replaced with fresh media. The monocytes that remain in the collagen matrix autonomously mature and differentiate into antigen-presenting cells (APCs) over about a day. One type of APC, macrophages, stays in the collagen matrix. The other type of APC, dendritic cells (DCs), migrates back through the collagen and endothelial cell layer and into the nutrient media. Once reverse transmigration of DCs has begun, the PTE cultures are ready to receive cell-bearing microcarrier beads.

It will be apparent to the skilled artisan that PTE cultures ready to receive cell-bearing microcarrier beads have populations of monocytes of varying maturation states. Such populations of monocytes include, but are not limited to, the following cell types: monocytes, macrophages, and dendritic cells. In certain embodiment of the invention, the populations of monocytes are of varying maturation states. In other embodiments of the invention, the populations of monocytes express a detectable level of one or more of the dendritic cell maturation markers selected from the group consisting of HLA-DR, CD14, CD86, and CCR7. The skilled artisan will understand that the dendritic cells can also be considered to comprise two sub-populations, namely immature and mature dendritic cells.

Upon addition of the cell-bearing microcarrier beads to the PTE cultures, thereby forming the co-cultures of the present invention, the effect of the test agents on the particular cell type attached to the microcarrier beads can be assayed. For example, in one test the cell-bearing microcarrier beads can be primed with bacterial proteins before addition to the PTE culture. The effect of the bacterial proteins on the bead-bound cells can be evaluated by assaying for a response by the cells comprising the populations of monocytes of varying maturation states in the co-culture, such as dendritic cells. The response can be assayed whether triggered by cell-cell contact between the bead-bound cells and cells in the culture, such as dendritic cells, or a release of factors from the bead-bound cells that are taken up by the cells in the culture, such as dendritic cells. These results can be compared to the response of cells in a co-culture in a control experiment where cell-bearing microcarrier beads are primed with media alone. Similarly, these results can form a baseline cell response in experiments designed to study the effects of a compound, such as a microbicide, on the ability of the bead-bound cells to resist bacterial or viral infection. In such experiments, the bead-bound cells can be treated with a compound, such as a microbicide, in advance of exposure to bacteria proteins. As will be apparent, in preferred aspects of the methods of the present invention, the cellular response that is assayed in a response by dendritic cells.

The cellular response of cells in the population of monocytes of varying maturation present in the co-culture can be assayed through a variety of techniques. In general, the cellular response is a parameter that includes, but is not limited to cellular maturation, cellular growth rate, cell number, apoptosis, cytokine production, chemokine production, and cellular marker expression. Cellular maturation includes maturation of dendritic cells present in the population of monocytes of varying maturation states. Cellular marker expression includes dendritic cell maturation marker expression where, the markers include, but are not limited to, one or more of HLA-DR, CD14, CD86, and CCR7. Cytokine production includes, but is not limited to production of one or more of IL-1a, IL-1b, IL-6, IL-8, IL-10, and TNF-α, such as by dendritic cells.

### EXAMPLES

Overall, ˜80-90% of urinary tract infections are caused by E. coli (Funfstuck, 2006). Over the past two decades, there has been an increase in reported cases of antimicrobial treatment-resistant E. coli strains in urinary tract infections (Aboderin, 2009). UTI infections have been linked to infertility through reproductive tract infections (Pallati, 2008; Foxman, 2002), pylonephritis or renal disease (Funfstuck, 2006), and prostatitis (Lipsky, 1999; Weidner, 2003) in the male population. Thus, E. coli was selected as an example bacterial antigen in co-culture models of the present invention.

### Example 1

### Summary of the Technique

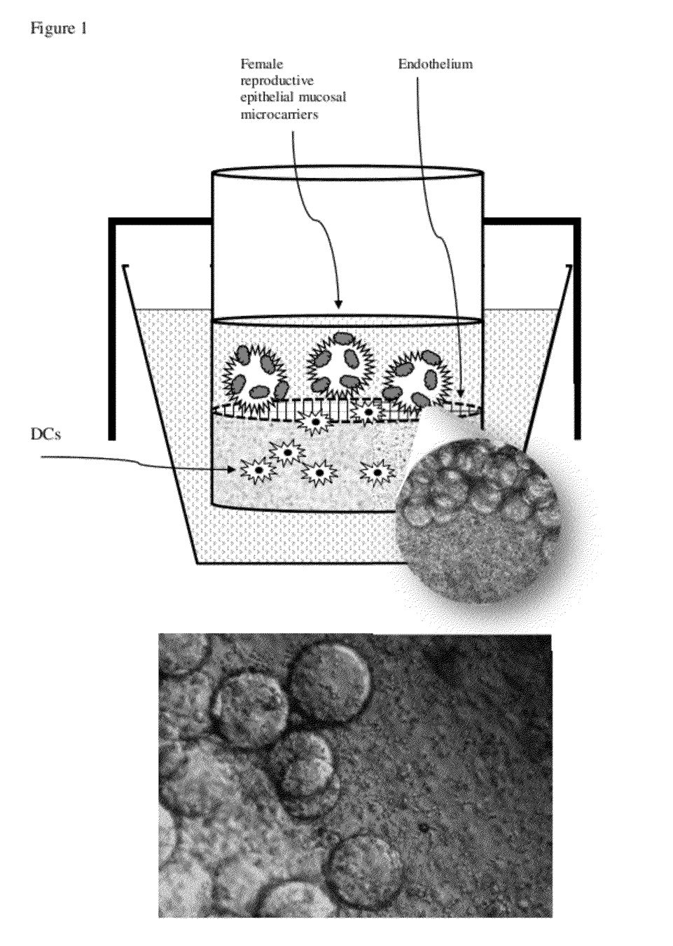

Our experimental design of the MIMIC® co-culture of RWV bioreactor-grown epithelial tissue from selected female reproductive tract tested the efficacy of the differentiated epithelia to pick up and present antigen (FIG. 1). RWV-cultured female reproductive microcarriers of differentiated vaginal and endocervical mucosa were exposed to microbicide/spermicide and bacterial antigen and then introduced to the APC-containing PTE module to study the innate immune responses to Nonoxynol-9 and E. coli bioparticulate treatments.

### Production of Vaginal Tissue Microcarriers and Endocervical Tissue Microcarriers

Approximately 3.5-week cultured vaginal tissue aggregates and endocervical tissue aggregates were used to mimic the local mucosal tissue of the female reproductive vaginal and endocervical regions. Cell-bearing microcarrier beads were prepared as follows. Collagen-coated Cytodex beads (Sigma) composed of dextran and having an average size of ˜60-87 microns were prepared for cell attachment by soaking in sterile PBS overnight and autoclaved. Once prepared, the microcarrier beads were added to a rotating-wall vessel (RWV) comprising KSFM complete media (Invitrogen) supplemented with primocin (InvivoGen) and ˜1×10⁷ vaginal epithelial cells were added to prepare vaginal tissue aggregates. Separately, ˜1×10⁷ endocervical epithelial cells were added to a RWV containing the collagen-coated microcarriers to prepare endocervical tissue aggregates. The microcarrier beads and cells were cultured in the RWV at a rate of approximately 20 rotations/min under conditions of 5% CO₂ and 37° C. for an initial 96-h period and then re-fed every day as needed for ˜3.5 weeks.

Successful preparation of vaginal tissue aggregates was determined using light and confocal microscopy and cell viability was tested using trypan blue exclusion staining. Successful preparation of endocervical tissue aggregates was determined similarly. In this example, collagen-coated microcarriers were used because they enhanced the surface area of the microcarrier beads and provided extracellular matrix necessary for the differentiation of the in vitro-grown epithelium. The RWV-derived aggregate cultures are amenable to shipment across the United States to be used in subsequent experiments and downstream assays. See generally Hjelm et al. (2010) Biol. Reprod. 82, 617-27.

### Production of PTE Modules

A detailed description of the peripheral tissue equivalent (PTE)/vaccination site (VS) module of the MIMIC® system is provided in U.S. Pat. No. 7,855,074.

Specifically, for the differentiated reproductive epithelial microcarrier co-cultures used here, we generated PTE modules over a period of 2 weeks as follows (see FIG. 1). Modules were prepared using a 4% collagen matrix gel with the addition of cell adhesion proteins, such as fibronectin, vitronectin, and laminin, layered on, for example, the membrane of a Transwell® (Corning) device (see FIG. 1), or simply on the base of the wells of a multi-well plate. Primary human umbilical vascular endothelial cells (HUVECs) added and cultured at a density of 30,000 to 200,000 cells per well. A 24-well format of the PTE was used for this experiment. A similar approach can be used with, for example, 96-, 48-, 12-, and 6-well formats. The cells were cultured in up to 35% fetal bovine serum containing Media199 (Sigma) with or without added antibiotics (penicillin+streptomycin+gentamycin; Sigma-Aldrich). Modules were cultured on a rocking platform at a rate of 15 min⁻¹. The rocker appeared to improve cell-cell contact and promoted the extravasation process. Non-adherent cells were washed off 2 h after addition of the HUVECs to the modules. The modules were cultured for about 2 weeks before further use. Cell layer integrity and viability were assessed by phase contrast microscopy and/or trypan blue exclusion staining.

### Preparing PTE Modules to Accept Cell-Bearing Microcarriers

Purified CD33⁺ monocytes were obtained by magnetic sorting (Miltenyi) of peripheral blood monocytes from female donors. This method depletes the cell suspension of T, B, and NK cells and thus limits confounding factors related to donor mismatch between the tissue and monocytes. Purity of the isolated cells was in the range of ˜95-100%. Purified monocytes were applied to the 2 week-cultured PTE modules to generate non-stimulated antigen-presenting cells (APCs). Cell densities of ˜0.5-30 million per well can be used in small- to large-scale PTE platforms. For consistency, reverse-transmigrated cells were washed off after ˜90 min of incubation. The surface area of the multi-well plate determines the density of monocyte entry through the confluent endothelium.

After removal of reverse-transmigrated cells, the modules were placed on a rocking platform (15 min⁻¹) to await receipt of populations of cell-bearing microcarriers. Prior to addition to the PTE modules, vaginal- and endocervix-cell bearing microcarriers were collected from the RWV, washed, and re-suspended in culture medium.

In some experiments, antigen-primed and/or microbicide-treated cell-bearing microcarriers were used, prepared as described below.

### Priming Cell-Bearing Microcarriers

Vaginal- and endocervix-cell bearing microcarriers were collected, washed, and re-suspended in 30 mL of KSFM complete medium (Invitrogen, with EGF and BPE), CaCl, and Primocin (InvivoGen). They were then placed on a shaker in polystyrene bottles with vented caps in an incubator (5% CO₂, 37° C.) to mimic the circular agitation of the RWV bioreactor. The two types of cell-bearing microcarriers were primed using E. coli as the antigen to mimic an in vivo infection of the female reproductive mucosa through the following steps after a day of culture in the incubator. The E. coli was heat-inactivated and the resulting bioparticles were added at a concentration of 1×10⁶/mL to the shaker bottles. E. coli bioparticles alone and no-treatment conditions served as positive and baseline controls, respectively.

The effect of the microbicide nonoxynol-9 (N-9) on antigen priming was studied in experiments by mimicking in vivo microbicidal exposure through the use of Conceptrol®, leading to tissue damage, followed by bacterial infection. Over-the-counter, N-9-containing Conceptrol® was diluted to 20 μg/mL. The concentration of Conceptrol® was chosen to limit massive cell death due to presence of N-9 and reported findings of weakened membrane integrity in the presence of this surfactant microbicide (Dayal, 2003). Experimental conditions included treatment with Conceptrol®, and in the presence or absence of heat-inactivated E. coli bioparticles. Aliquots of the two types of microcarriers were treated with 20 μg/mL N-9 for 4 h under conditions of 35% serum-containing M199 prior to introduction of E. coli. On addition of E. coli, the cultures were cultured for an additional 4 h. Controls including cultures with E. coli antigen alone or N-9 alone were used to isolate the response to bacterial infection, to model urinary tract infections that typically spread to the reproductive mucosa.

The negative (no antigen) controls of PTE alone and PTE plus cell-bearing microcarriers (discussed below) provided a baseline level to aid in dissecting the effects of treatment and minimizing confounding factors from the presence of multiple cell types.

### Co-Culture Testing

After selected treatments and/or priming, the different populations of reproductive tissue cell-bearing microcarriers were added to PTE modules to create co-cultures. Co-cultures were incubated overnight on a rocking platform (15 min⁻¹, 5% CO₂, 37° C.). Cellular maturation and cytokine responses were used to characterize the innate immune responses to microbicide and bacterial antigens.

A Bioplex®-based assay was used to measure cytokine production in the co-culture supernatants at 24 h incubation with the primed microcarriers.

The quality of immune APCs was determined by quantifying dendritic cell marker expression (HLA-DR, CD14, CD86, CCR7) from the reverse-transmigrated cell fraction of the MIMIC® co-cultures.

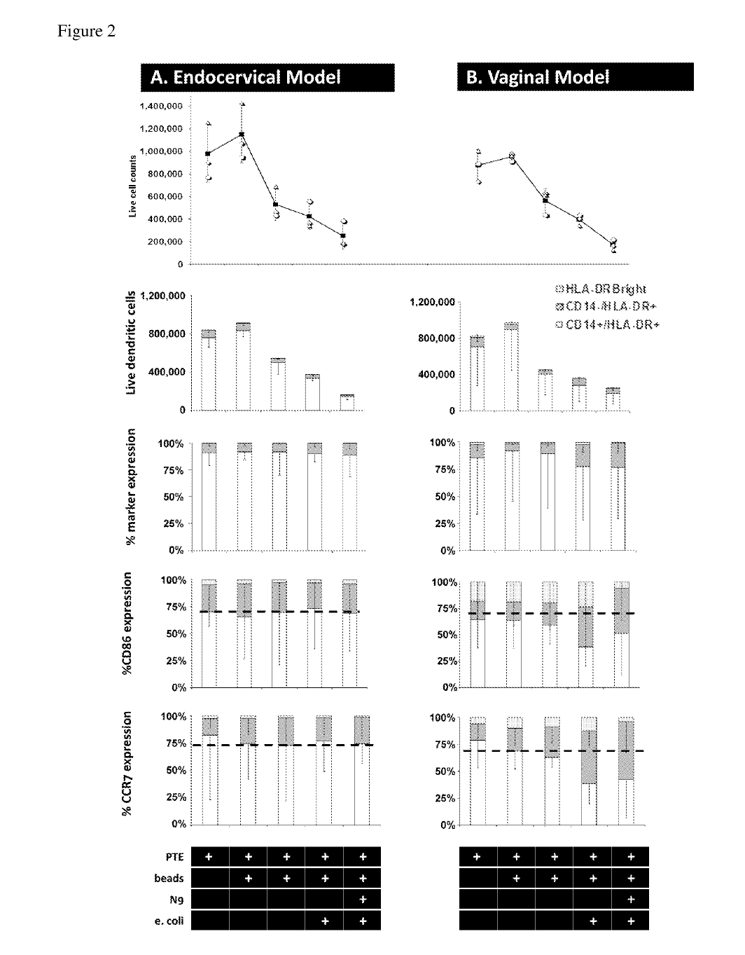

The innate immune response data for the co-cultures showed an overall reduction in live dendritic cell recovery after treatment with N-9, E. coli bioparticles, or both delivered together in both the endocervical and vaginal models (FIG. 2). The degree of APC and cytokine production was greater in the vaginal model.

The quality of antigen-presenting cells was assessed by DC maturation marker expression (CD14, HLA-DR, CD86, CCR7). DC marker expression in the PTE and microcarrier co-cultures of the multi-layered vaginal epithelium showed more efficient uptake of antigen versus the endocervical counterparts.

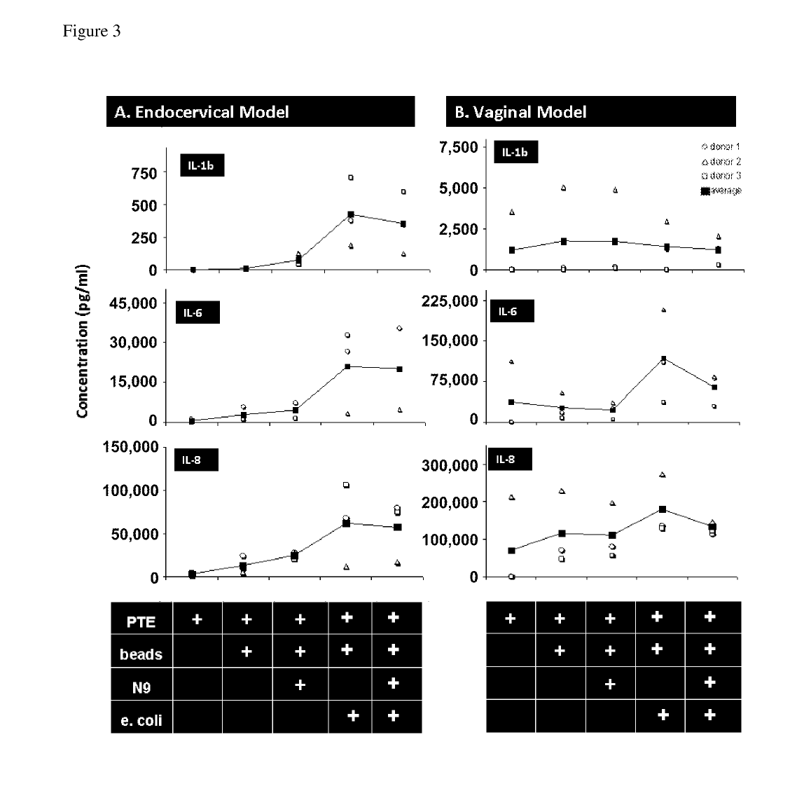

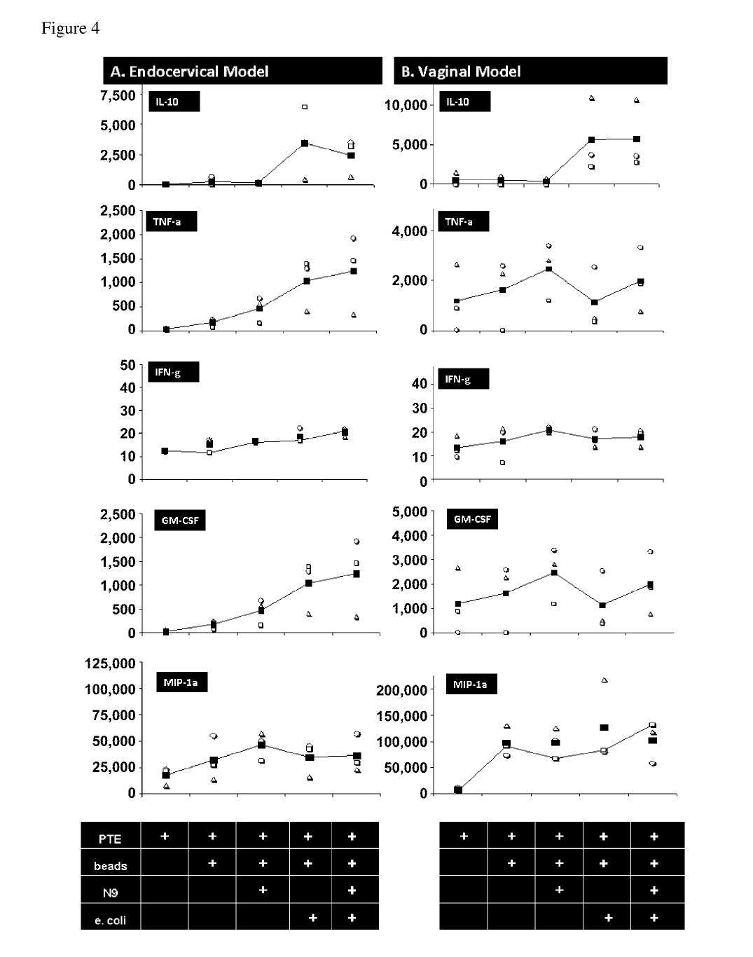

FIGS. 3 and 4 show pro-inflammatory cytokine profiles for example MIMIC® co-culture experiments. Generally, the cytokine levels for the vaginal model were several-fold higher than the endocervical model. This may be due to an epithelial cell population from the multi-layered tissue and, thus, a higher background. However, the response generated by the endocervical model was typically more consistent, where most of the cytokines produced were above the levels in the no-treatment controls. Pro-inflammatory cytokine (IL-1b, IL-6, IL-8) up-regulation by the introduction of E. coli particulates is consistent with literature findings of bacteria-induced innate responses by mucosal tissue (Bassett, 2003; Bergsten, 2005). Interleukin-10 and TNF-α levels also increased with E. coli treatment, consistent with reported findings (Basset, 2003).

The cytokine response generated by the example vaginal model closely resembled in vitro mRNA expression of IL-8, TNF-α, and IL-1a in an immortalized vaginal cell line, PK/E6/E7 (Pivarcsi, 2005). The cytotoxic nature of N-9 and pro-inflammatory response in another vaginal immortalized cell line (VK-2) also resembled our findings (Doncel, 2004). The pro-inflammatory cytokine response has been suggested to be a key biomarker for determining the efficacy of microbicides in pre-clinical and clinical trials (Fichorova R N, 2004).

Overall, our preliminary findings of innate immune responses in the PTE and female reproductive microcarrier co-culture are consistent with cell lines and limited in vivo findings. However, our co-culture data showed a more robust immune response compared with previous in vitro models, because the present invention includes immune cell responses using primary human cells in a culture system without stimulants. Overall, these examples suggest that co-culture model systems of the present invention successfully replicate the in vivo situation.

All documents, publication, manuals, article, patents, summaries, references and other materials cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

### REFERENCES

Hammond T G, Hammond J M. Optimized suspension culture: the rotating-wall vessel. Am J Physiol Renal Physiol. 2001 July; 281(1):F12-25.
Goodwin T J, Schroeder W F, Wolf D A, Moyer M P. Rotating-wall vessel co-culture of small intestine as a prelude to tissue modeling: aspects of simulated microgravity. Proc Soc Exp Biol Med. 1993 February; 202(2):181-92.
O'Connor K C. Three-dimensional cultures of prostatic cells: tissue models for the development of novel anti-cancer therapies. Pharm Res. 1999 April; 16(4):486-93.
Goodwin T J, Prewett T L, Spaulding G F, Becker J L. Three-dimensional culture of a mixed Mullerian tumor of the ovary: expression of in vivo characteristics. In Vitro Cell Dev Biol Anim. 1997 May; 33(5):366-74.
Hjelm B E, Berta A N, Nickerson C A, Arntzen C J, Herbst-Kralovetz M M. Development and characterization of a three-dimensional organotypic human vaginal epithelial cell model. Biol Reprod. 2010 March; 82(3):617-27.
Mitteregger R, Vogt G, Rossmanith E, Falkenhagen D. Rotary cell culture system (RCCS): a new method for cultivating hepatocytes on microcarriers. Int J Artif Organs 1999 December; 22(12):816-22.
Duke P J, Daane E L, Montufar-Solis D. Studies of chondrogenesis in rotating systems. J Cell Biochem. 1993 March; 51(3):274-82.
Mikaty G, Soyer M, Mairey E, Henry N, Dyer D, Forest K T, Morand P, Guadagnini S, Prévost MC, Nassif X, Duménil G. Extracellular bacterial pathogen induces host cell surface reorganization to resist shear stress. PLoS Pathog. 2009 February; 5(2):e1000314.
Andrei G. Three-dimensional culture models for human viral diseases and antiviral drug development. Antiviral Res. 2006 September; 71(2-3):96-107.
Nauman E A, Ott C M, Sander E, Tucker D L, Pierson D, Wilson J W, Nickerson C A. Novel quantitative biosystem for modeling physiological fluid shear stress on cells. Appl Environ Microbiol. 2007 February; 73(3):699-705.
Simons D M, Gardner E M, Lelkes P I. Dynamic culture in a rotating-wall vessel bioreactor differentially inhibits murine T-lymphocyte activation by mitogenic stimuli upon return to static conditions in a time-dependent manner. J Appl Physiol. 2006 April; 100(4): 1287-92.
Borchers A T, Keen C L, Gershwin M E. Microgravity and immune responsiveness: implications for space travel. Nutrition 2002 October; 18(10):889-98.
Cogoli A. Space flight and the immune system. Vaccine 1993; 11(5):496-503.
Fitzgerald W, Chen S, Walz C, Zimmerberg J, Margolis L, Grivel J C. Immune suppression of human lymphoid tissues and cells in rotating suspension culture and onboard the International Space Station. In Vitro Cell Dev Biol Anim. 2009 Jul. 16.
Ritz B W, Lelkes P I, Gardner E M. Functional recovery of peripheral blood mononuclear cells in modeled microgravity. FASEB J. 2006 February; 20(2):305-7.
Cherry R S, Papoutsakis E T. Physical mechanisms of cell damage in microcarrier cell culture bioreactors. Biotechnol Bioeng. 1988 Oct. 5; 32(8):1001-14.
Higbee R G, Byers A M, Dhir V, Drake D, Fahlenkamp H G, Gangur J, Kachurin A, Kachurina O, Leistritz D, Ma Y, Mehta R, Mishkin E, Moser J, Mosquera L, Nguyen M, Parkhill R, Pawar S, Poisson L, Sanchez-Schmitz G, Schanen B, Singh I, Song H, Tapia T, Warren W, Wittman V. An immunologic model for rapid vaccine assessment—a clinical trial in a test tube. Ahern Lab Anim. 2009 September; 37 Suppl 1:19-27.
Ma Y, Poisson L, Sanchez-Schmitz G, Pawar S, Qu C, Randolph G J, Warren W L, Mishkin E M, Higbee R G. Assessing the immunopotency of Toll-like receptor agonists in an in vitro tissue-engineered immunological model. Immunology 2010 Mar. 17
Byers A M, Tapia T M, Sassano E R, Wittman V. In vitro antibody response to tetanus in the MIMIC system is a representative measure of vaccine immunogenicity. Biologicals 2009 June; 37(3):148-51
Foxman B. Epidemiology of urinary tract infections: incidence, morbidity, and economic costs. Am J Med. 2002 Jul. 8; 113 Suppl 1A:5S-13S.
Foxman B, Frerichs R R. Epidemiology of urinary tract infection: II. Diet, clothing, and urination habits. Am J Public Health 1985 November; 75(11): 1314-1317.
Fünfstück R, Ott U, Naber K G The interaction of urinary tract infection and renal insufficiency. Int J Antimicrob Agents 2006 August; 28 Suppl 1:S72-7.
Aboderin O A, Abdu A R, Odetoyin B W, Lamikanra A. Antimicrobial resistance in Escherichia coli strains from urinary tract infections. J Natl Med Assoc. 2009 December; 101(12): 1268-73.
Pellati D, Mylonakis I, Bertoloni G, Fiore C, Andrisani A, Ambrosini G, Armanini D. Genital tract infections and infertility. Eur J Obstet Gynecol Reprod Biol. 2008 September; 140(1):3-11.
Lipsky B A Prostatitis and urinary tract infection in men: what's new; what's true? Am J Med. 1999 March; 106(3):327-34.
Weidner W, Ludwig M. Common Organisms in Urogenital Infections with special impact on prostatitis. European Urology Supplements 2 (2003) 15-18.
Lederman M M, Offord R E, Hartley O. Microbicides and other topical strategies to prevent vaginal transmission of HIV. Nat Rev Immunol. 2006 May; 6(5):371-82.
Cone R A, Hoen T, Wong X, Abusuwwa R, Anderson D J, Moench T R. Vaginal microbicides: detecting toxicities in vivo that paradoxically increase pathogen transmission. BMC Infect Dis. 2006 Jun. 1; 6:90.
Dayal M B, Wheeler J, Williams C J, Barnhart K T. Disruption of the upper female reproductive tract epithelium by Nonoxynol-9. Contraception 2003 October; 68(4):273-9
Kiyono H, Miller C J, Lu Y, Lehner T, Cranage M, Huang Y T, Kawabata S, Marthas M, Roberts B, Nedrud J G, Lamm M E, Bergmeier L, Brookes R, Tao L and McGhee_JR. The common mucosal immune system for the reproductive tract: basic principles applied toward an AIDS vaccine. Advanced Drug Delivery Reviews Volume 18, Issue 1, 15 Dec. 1995, Pages 23-52.
Basset C, Holton C, O'Mahony R, Roitt I. Innate immunity and pathogen-host interaction. Vaccine 2003 Jun. 1; 21 Suppl 2:S12-23.
Bergsten G, Wullt B, Svanborg C. Escherichia coli, fimbriae, bacterial persistence and host response induction in the human urinary tract. Int J Med Microbiol. 2005 October; 295(6-7): 487-502.
Pivarcsi A, Nagy I, Koreck A, K is K, Kenderessy-Szabo A, Szell M, Dobozy A, Kemeny L Microbial compounds induce the expression of pro-inflammatory cytokines, chemokines and human beta-defensin-2 in vaginal epithelial cells. Microbes Infect. 2005 July; 7(9-10): 1117-27.
Doncel G F, Chandra N, Fichorova R N. Preclinical assessment of the proinflammatory potential of microbicide candidates. J Acquir Immune Defic Syndr. 2004 October; 37 Suppl 3: S174-80.
Fichorova R N. Guiding the vaginal microbicide trials with biomarkers of inflammation. J Acquir Immune Defic Syndr. 2004 October; 37 Suppl 3:S184-93.
Hjelm B E, Berta A N, Nickerson C A, Arntzen C J, Herbst-Kralovetz M M. Development and characterization of a three-dimensional organotypic human vaginal epithelial cell model Biol Reprod. 2010 March; 82(3):617-27.

What is claimed is:

1. A method of assaying a cellular response to a test agent in an in vitro urogenital co-culture, said method comprising:
a) priming epithelial cell-bearing microcarrier beads with one or more test agents, wherein the microcarrier beads are (i) vaginal epithelial cell-bearing microcarrier beads, or (ii) endocervical epithelial cell-bearing microcarrier beads;
b) adding the primed microcarrier beads of a) to a peripheral tissue equivalent (PTE) culture, thereby preparing a co-culture, wherein the PTE culture comprises primary human umbilical vascular endothelial cells (HUVECs) attached to a substantially planar matrix and a population of monocytes of varying maturation states; and
c) assaying a cellular response to the test agent in the co-culture of b), thereby assaying a cellular response to a test agent in an in vitro urogenital co-culture.

2. The method of claim 1, further comprising comparing the response assayed in c) to the response assayed in a counterpart control co-culture in which the microcarrier beads were not primed with the one or more test agents.

3. The method of claim 1, wherein the one or more test agent is selected from the group consisting of bacteria, viruses, environmental pollutants, vaccines, adjuvants, immunotherapy candidates, cosmetics, drugs, biologics, proteins, and chemical compounds.

4. The method of claim 1, wherein the test agent is a species or strain of bacteria.

5. The method of claim 1, wherein the test agent is a chemical compound containing nonoxynol-9.

6. The method of claim 1, wherein the test agent is a species or strain of bacteria and a chemical compound containing nonoxynol-9.

7. The method of claim 1, wherein the cells of the PTE culture consist of HUVECs and a population of monocytes of varying maturation states.

8. The method of claim 1, wherein the population of monocytes of varying maturation states expresses a detectable level of one or more of the dendritic cell maturation markers selected from the group consisting of HLA-DR, CD14, CD86, and CCR7.

9. The method of claim 2, wherein the population of monocytes of varying maturation states expresses a detectable level of one or more of the dendritic cell maturation markers selected from the group consisting of HLA-DR, CD14, CD86, and CCR7.

10. The method of claim 1, wherein the matrix is selected from the group consisting of collagen, a collagen gel, a hydrogel, PLA, PLGA, gelatin, hyaluronic acid, a synthetic material, and a combination thereof.

11. The method of claim 10, wherein the matrix is a collagen gel, optionally further comprising one or more cell adhesion proteins selected from the group consisting of fibronectin, vitronectin, and laminin.

12. The method of claim 1, wherein the cellular response is a parameter selected from the group consisting of cellular maturation, cellular growth rate, cell number, apoptosis, cytokine production, chemokine production, and cellular marker expression.

13. The method of claim 1, wherein the cellular response is cellular maturation or cytokine production, or both.

14. The method of claim 2, wherein the cellular response is cellular maturation or cytokine production, or both.

15. The method of claim 13, wherein the cellular maturation is maturation of dendritic cells present in the population of monocytes of varying maturation states.

16. The method of claim 15, wherein cellular maturation of dendritic cells is assayed by screening for expression of one or more dendritic cell maturation markers selected from the group consisting of HLA-DR, CD14, CD86, and CCR7.

17. The method of claim 13, wherein cytokine production is production of one or more cytokines selected from the group consisting of IL-1a, IL-1b, IL-6, IL-8, IL-10, and TNF-α.

18. The method of claim 1, wherein the microcarrier beads of the vaginal epithelial cell-bearing microcarrier beads and endocervical epithelial cell-bearing microcarrier beads are dextran beads.

19. The method of claim 1, wherein the microcarrier beads of the vaginal epithelial cell-bearing microcarrier beads and endocervical epithelial cell-bearing microcarrier beads are collagen-coated dextran beads.

20. The method of claim 1, wherein the vaginal epithelial cell-bearing microcarrier beads and endocervical epithelial cell-bearing microcarrier beads are prepared in a rotating-wall vessel bioreactor.
